(19) United States
(12) Patent Application Publication
(10) Pub. No.: US 2008/0107733 A1
(43) Pub. Date: May 8, 2008

(54) Formulation Obtained From A Powder Mixture Comprising An Inorganic Pigment

(76) Inventors: Katja Fastnacht, Mannheim (DE); Matthias Degenhardt, Ludwigshafen (DE); Gunther Berndl, Herxheim (DE); Jorg Rosenberg, Ellerstadt (DE); Jorg Breitenbach, Mannheim (DE)

Correspondence Address: EDWARDS ANGELL PALMER & DODGE LLP, P.O. BOX 55874, BOSTON, MA, 02205, US

(21) Appl. No.: 11/597,799

(22) PCT Filed: May 30, 2005
(86) PCT No.: PCT/EP05/05806

§ 371 Date: Aug. 28, 2007

(30) Foreign Application Priority Data
May 28, 2004 (EP) 04012791.2

(51) Int. Cl. A61K 9/14

(52) U.S. Cl. 424/484

(57) ABSTRACT

A solid dosage form having a matrix with at least one active compound dispersed homogeneously in the matrix, which is obtainable by melting a powder mixture, wherein the powder mixture comprises at least one thermoplastic binder and a combination of highly disperse silica and at least one inorganic pigment, is described. The co-use of the highly disperse silica and the inorganic pigments leads to better flow properties of the powder mixture, has the effect of a faster release of the active compound from the dosage forms obtained, and imparts a visually pleasing appearance to the dosage forms. A process for the preparation of the dosage form is also described.

The present invention relates to a solid dosage form which is obtainable from a powder mixture comprising a combination of highly disperse silica and an inorganic pigment, and to a process for the preparation of the dosage form. The preparation of solid dosage forms by melt extrusion, i.e. a process in which a melt of a polymeric binder and an active compound is extruded and the extruded strand is shaped into the desired medicament form, is known, see e.g. EP-A 240 904, EP-A 240 906, EP-A 337 256 and EP-A 358 105. This process allows the preparation of sparingly soluble active compounds in the form of solid solutions. The active compound is present in the solid solutions in molecularly dissolved form and can therefore be absorbed more easily than the crystalline active compound.

During the extrusion, the constituents of the melt are exposed briefly to a not inconsiderable thermal stress, which can lead to a very slight decomposition of the polymeric binder and/or the other constituents and to a discoloration of the extrudate. The discolorations are indeed acceptable, but they impair the visual appearance of the dosage forms obtained. The discolorations are conventionally masked by application of a film coating to the dosage form, e.g. by spraying on a solution of a film-forming polymer. However, due to the contact with water or another solvent during the spraying on of the coating solution, partial recrystallization of the active compound may be induced on the surface of the dosage form, with the result that the release properties of the dosage form are changed in an uncontrolled manner.

In melt extrusion, a powder mixture of the binder, the active compound and optionally further components is introduced into the feed hopper of a suitable kneader or extruder. Good flow properties of the powder mixture are desirable for a problem-free preparation and an accurate volumetric metering. However, the powder mixtures often tend towards the formation of bridges, because of adhesion of the individual binder particles to one another, and non-uniform flow properties. Zimmermann, I. et al. report in Powder Technology 139 (2004) 40-54 on the influence of various flow auxiliaries on the flow properties of maize starch.

WO 92/15285 describes depot formulations which comprise a processed starch. The starch is processed in an extruder under shear conditions at temperatures of from 80 to 240° C. Fillers, such as titanium dioxide, can be co-used here.

U.S. Pat. No. 5,427,614 discloses starch formulations for injection molding which comprise starch, a lubricant, a flow agent, a texturizing agent and water. Combinations of silicon dioxide and titanium dioxide e.g. are regarded as texturizing agents. The starch formulations contain no active compound; a use of the starch formulations as dosage forms is not envisaged.

The invention is based on the object of avoiding the melt extrusion problems described above. In particular, the invention is based on the object of providing a dosage form which can be prepared by melt extrusion, which has a rapid release of the active compound, which has a visually pleasing appearance without requiring a film coating and can be prepared in an efficient manner.

It has now been found that the co-use of inorganic pigments in the powder mixture not only leads to better flow properties of the powder mixture and therefore to better intake properties in the feed hopper. Surprisingly, the co-use of the inorganic pigments also has the effect of a faster release of the active compound from the dosage forms obtained, and imparts to the dosage forms a visually pleasing appearance, also without an additional film coating.

The present invention provides a solid dosage form having a matrix with at least one active compound dispersed homogeneously in the matrix, which is obtainable by melting a powder mixture, wherein the powder mixture comprises at least one thermoplastic binder and a combination of highly disperse silica and at least one inorganic pigment.

The active compound is preferably present in the dosage form according to the invention substantially in an amorphous form, recognizable from the absence of diffraction maxima in the X-ray diffraction spectrum which are to be attributed to the crystalline active compound. The active compound is particularly preferably present in the matrix in the form of a solid solution.

The powder mixture for the preparation of preferred dosage forms comprises:

1.0 to 30 wt. % (preferably 2.5 to 20 wt. %) of active compound,
0 to 20 wt. % (preferably 0.1 to 15 wt. %) of solubilizer,
0.5 to 2.0 wt. % (preferably 0.8 to 1.5 wt. %) of highly disperse silica,
0.8 to 5.0 wt. % (preferably 1.0 to 4.0 wt. %) of inorganic pigment, and
45 to 97 wt. % of thermoplastic binder.

The invention furthermore relates to a process for the preparation of solid dosage forms, in which

a) a powder mixture which comprises at least one thermoplastic binder and a combination of highly disperse silica and at least one inorganic pigment is provided,
b) the powder mixture is melted, and
c) the melt obtained is shaped into dosage forms.

The angle of repose is a measure of the flowability of a powder mixture. The angle of repose is the angle between the horizontal plane and the taper which is established when bulk material is poured on to this plane. One possibility for determining the angle of repose is to fill a vessel with the powder mixture and then to remove a side wall. It is likewise possible to collect the powder mixture in a rotating drum and then to measure the angle which the powder surface encloses with the horizontal plane. Preferably, however, the angle of repose is determined by means of a Dr. Pfrengle angle of repose measuring apparatus. A funnel having an top internal diameter of 140 mm and an outflow internal diameter of 10 mm is attached to a stand. The funnel comprises a crank and a stirring hoop to force powder which does not flow freely out of the funnel opening. A base plate having a diameter of 100 mm is arranged centrally under the funnel outflow. The funnel is filled with the powder to be tested. In the case of free-flowing powders, the outflow opening is released; in the case of forced-flow powder, the opening is released and the crank of the stirring hoop is turned uniformly. An amount of powder sufficient for the poured cone which forms to cover the base plate completely is allowed to flow out of the funnel. The height of the powder cone is then determined with a measuring rod. The angle of repose α is calculated according to the following equation:

tan α=h/r

wherein h is the height of the powder cone and r is the radius of the base plate (50 mm). The measurement is repeated twice and the mean of the three individual measurements is obtained.

As the following examples and comparison examples show, the angle of repose of the powder mixture is reduced drastically by the co-use of a combination of highly disperse silica and at least one inorganic pigment. The reasons for this have not been completely clarified; the effect observed is presumably based on the one hand on the increase in the bulk density of the powder mixture due to the pigment of high specific gravity. On the other hand, the particles of the silica and/or of the inorganic pigment attach themselves to the surface of the larger binder particles and increase the surface roughness thereof. As a result, the binder particles can no longer come so close to one another that adhesion forces can develop between the individual particles.

Preferably, the angle of repose of a powder mixture used according to the invention which comprises a binder in the form of a copolymer of 60 wt. % of vinylpyrrolidone and 40 wt. % of vinyl acetate is less than 39°, preferably less than 38.5°, in particular less than 37°.

Preferably, the powder mixture has a particle size distribution wherein at least 50 wt. % of the particles have a particle size of less than 200 μm and at least 10 wt. % of the particles have a particle size of less than 100 μm. In particular, at least 80 wt. % of the particles have a particle size of less than 200 μm and at least 30 wt. % of the particles have a particle size of less than 100 μm.

The amount of inorganic pigment contained in the powder mixture is preferably tinctorially active. For the purpose of the present Application, the expression “tinctorially active” is intended to means that the colored appearance of dosage forms according to the invention can be distinguished visually from that of such dosage forms which are prepared in an identical manner from a powder mixture which is otherwise identical but without an inorganic pigment. Colors to which different color numbers are assigned in the PANTONE color definition system (Pantone Matching System®) e.g. are regarded as “visually distinguishable”.

The nature of the inorganic pigment is not particularly critical, as long as it is a pharmaceutically acceptable inorganic pigment which is inert towards the other components of the dosage form. The inorganic pigment is preferably chosen from titanium dioxide, in particular the anatase modification thereof, and iron oxides, such as goethite (yellow iron oxide), hematite (red iron oxide) and magnetite (black iron oxide). Those pigments which are approved as foodstuffs additives, e.g. under number E 172 of the list of approved foodstuffs additives of the European Union, are suitable in particular. The average particle size (determined by laser diffraction) of the inorganic pigment is as a rule less than 50 μm, preferably less than 10 μm.

The powder mixture as a rule comprises 0.8 to 5.0 wt. %, preferably 1 to 4.0 wt. % of inorganic pigment, based on the total weight of the powder mixture.

Highly disperse silica is extremely finely divided silicon dioxide, which is prepared by high temperature hydrolysis. The primary particle size is as a rule less than 50 nm. Suitable highly disperse silicas are, in particular, those having a BET surface area of from 100 to 400 m²/g, preferably 175 to 225 m²/g. A particularly suitable highly disperse silica is obtainable under the name Aerosil® 200.

Dosage forms are to be understood as meaning all forms which are suitable for use as medicaments, in particular for oral (especially peroral) administration, plant treatment agents, feedstuffs and foodstuff supplements. These include, for example, tablets of any form, capsules, pellets or granules, and films and foils.

The powder mixture comprises at least one thermoplastic binder. This is as a rule chosen from sugar alcohols, derivatives thereof and water-soluble and water-dispersible polymers. The binder is in pulverulent form, the particles preferably having a weight-average particle size in the range of from 50 to 1,000 μm, in particular 100 to 500 μm. The thermoplastic binder typically makes up 20 to 95 wt. % of the powder mixture.

Suitable thermoplastic polymers are, for example,

polyvinylpyrrolidone (PVP),
copolymers of N-vinylpyrrolidone and vinyl acetate and/or vinyl propionate,
copolymers of vinyl acetate and crotonic acid,
partly saponified polyvinyl acetate, polyvinyl alcohol,
polyhydroxyalkyl acrylates, polyhydroxyalkyl methacrylates,
polyacrylates and polymethacrylates (Eudragit® types),
copolymers of methyl methacrylate and acrylic acid,
polyethylene glycols (macrogol), polyethylene glycol/polypropylene glycol copolymers (poloxamers),
alkylcelluloses, in particular methylcellulose and ethylcellulose,
hydroxyalkylcelluloses, in particular hydroxypropylcellulose (HPC),
hydroxyalkyl-alkylcelluloses, in particular hydroxypropylmethylcellulose (HPMC),
cellulose esters, such as cellulose phthalates, in particular cellulose acetate phthalate, hydroxypropylmethylcellulose phthalate and hydroxypropyl-methylcellulose acetate succinate (HPMCAS),
starch, starch ester.

Binders which differ from starch and starch derivatives, such as native starch, degraded starch, preglutinized starch or starch esters, are preferably used.

Of these, homo- or copolymers of vinylpyrrolidone are particularly preferred, e.g. polyvinylpyrrolidone having Fikentscher K values of from 12 to 100, preferably 17 to 30, or copolymers of from 30 to 70 wt. % of N-vinylpyrrolidone (VP) and 70 to 30 wt. % of vinyl acetate (VA), such as e.g. a copolymer of 60 wt. % of VP and 40 wt. % of VA (copovidone). Hydroxypropylcellulose is furthermore preferred.

The thermoplastic polymers preferably have a softening temperature of from 60 to 180° C., in particular 70 to 130° C.

Suitable sugar alcohols are sorbitol, xylitol, mannitol, maltitol; a suitable sugar alcohol derivative is isomaltol or hydrogenated condensed palatinose, as described in DE 102 62 005.

Mixtures of the binders mentioned can of course also be employed.

The powder mixture comprises at least one active compound. Active compounds in the context of the invention are to be understood as meaning all substances having a desired physiological action on the human or animal body or plants. They are, in particular, pharmaceutical active compounds. The amount of active compound per dose unit can vary within wide limits. It is as a rule chosen such that it is sufficient to achieve the desired action. Active compound combinations can also be employed. Active compounds in the context of the invention are also vitamins and mineral substances. The vitamins include the vitamins of the A group, of the B group, by which are also to be understood, in addition to B₁, B₂, B₆ and B₁₂ as well as nicotinic acid and nicotinamide, compounds having vitamin B properties, such as e.g. adenine, choline, pantothenic acid, biotin, adenylic acid, folic acid, orotic acid, pangamic acid, carnitine, p-aminobenzoic acid, myo-inositol and liponic acid, and vitamin C, vitamins of the D group, E group, F group, H group, I and J group, K group and P group. Active compounds in the context of the invention also include peptide therapeutics and proteins. Plant treatment agents include e.g. vinclozolin, epoxiconazole and quinmerac.

The process according to the invention is suitable, for example, for processing the following active compounds:

acebutolol, acetylcysteine, acetylsalicylic acid, aciclovir, albrazolam, alfacalcidol, allantoin, allopurinol, ambroxol, amikacin, amiloride, aminoacetic acid, amiodarone, amitriptyline, amlodipine, amoxicillin, ampicillin, ascorbic acid, aspartame, astemizole, atenolol, beclomethasone, benserazide, benzalkonium hydrochloride, benzocaine, benzoic acid, betamethasone, bezafibrate, biotin, biperiden, bisoprolol, bromazepam, bromhexine, bromocriptine, budesonide, bufexamac, buflomedil, buspirone, caffeine, camphor, captopril, carbamazepine, carbidopa, carboplatin, cefachlor, cefalexin, cefatroxil, cefazolin, cefixime, cefotaxime, ceftazidime, ceftriaxone, cefuroxime, celedilin, chloramphenicol, chlorhexidine, chlorpheniramine, chlortalidone, choline, cyclosporin, cilastatin, cimetidine, ciprofloxacin, cisapride, cisplatin, clarithromycin, clavulanic acid, clomibramine, clonazepam, clonidine, clotrimazole, codeine, cholestyramine, cromoglycic acid, cyanocobalamin, cyproterone, desogestrel, dexamethasone, dexpanthenol, dextromethorphan, dextropropoxiphene, diazepam, diclofenac, digoxin, dihydrocodeine, dihydroergotamine, dihydroergotoxin, diltiazem, diphenhydramine, dipyridamole, dipyrone, disopyramide, domperidone, dopamine, doxycycline, enalapril, ephedrine, epinephrine, ergocalciferol, ergotamine, erythromycin, estradiol, ethinylestradiol, etoposide, Eucalyptus globulus, famotidine, felodipine, fenofibrate, fenofibric acid, fenoterol, fentanyl, flavin mononucleotide, fluconazole, flunarizine, fluorouracil, fluoxetine, flurbiprofen, furosemide, gallopamil, gemfibrozil, gentamicin, Gingko biloba, glibenclamide, glipizide, clozapine, Glycyrrhiza glabra, griseofulvin, guaifenesin, haloperidol, heparin, hyaluronic acid, hydrochlorothiazide, hydrocodone, hydrocortisone, hydromorphone, ipratropium hydroxide, ibuprofen, imipenem, indomethacin, insulin, isohexyl, isopamidol, isosorbide dinitrate, isosorbide mononitrate, isotretinoin, itraconazol, ketotifen, ketoconazole, ketoprofen, ketorolac, labatalon, lactulose, lecithin, levocarnitine, levodopa, levoglutamide, levonorgestrel, levothyroxine, lidocaine, lipase, lipramine, lisinopril, loperamide, lopinavir, lorazepam, lovastatin, medroxyprogesterone, menthol, methotrexate, methyldopa, methylprednisolone, metoclopramide, metoprolol, miconazole, midazolam, minocycline, minoxidil, misoprostol, morphine, multivitamin mixtures and combinations and mineral salts, N-methylephedrine, naftidrofuryl, naproxen, neomycin, nicardipine, nicergoline, nicotinamide, nicotine, nicotinic acid, nifedipine, nimodipine, nitrazepam, nitrendipine, nizatidine, norethisterone, norfloxacin, norgestrel, nortriptyline, nystatin, ofloxacin, omeprazole, ondansetron, pancreatin, panthenol, pantothenic acid, paracetamol, penicillin g, penicillin v, phenobarbital, phenoxifylline, phenoxymethylpenicillin, phenylephrine, phenylpropanolamine, phenyloin, piroxicam, polymyxin b, povidone-iodine, pravastatin, prazepam, prazosin, prednisolone, prednisone, promocriptine, propafenone, propranolol, proxyphylline, pseudoephedrine, pyridoxine, quinidine, ramipril, ranitidine, reserpine, retinol, riboflavin, rifampicin, ritonavir, rutoside, saccharin, salbutamol, salcatonin, salicylic acid, simvastatin, somatropin, sotalol, spironolactone, sucralfate, sulbactam, sulfamethoxazole, sulfasalazine, sulpiride, tamoxifen, tegafur, teprenone, terazosin, terbutaline, terfenadine, tetracycline, theophylline, thiamine, ticlopidine, timolol, tranexamic acid, tretinoin, triamcinolone acetonide, triamterene, trimethoprim, troxerutin, uracil, valproic acid, vancomycin, verapamil, vitamin E, volic acid, zidovudine.

The process is particularly suitable for active compounds having a solubility in water at 25° C. of less than 1 mg/ml. Such active compounds are also described according to USP XXII, p. 8 as scarcely soluble or practically insoluble.

The active compound is as a rule in pulverulent form, the particles preferably having a weight-average particle size in the range of from 1 to 1,000 μm, in particular 5 to 850 μm. The active compound typically makes up 1 to 90 wt. %, preferably 5 to 60 wt. % of the powder mixture.

The powder mixture can additionally comprise various optional auxiliaries. Pulverulent auxiliaries can be simply mixed in. Liquid or paste-like auxiliaries can expediently be mixed beforehand with the amount of binder or a portion thereof, granules being obtained, which can be mixed in a simple manner with the remainder of the amount of binder and the other components of the powder mixture. Alternatively, the auxiliaries can be incorporated into the melt, e.g. via metering points in the barrel of the extruder.

Such optional auxiliaries are:

Solubilizers, such as sorbitan fatty acid esters, polyalkoxylated fatty acid esters, such as e.g. polyalkoxylated glycerides, polyalkoxylated sorbitan fatty acid esters or fatty acid esters of polyalkylene glycols; or polyalkoxylated ethers of fatty alcohols. A fatty acid chain in these compounds as a rules comprises 8 to 22 carbon atoms. The polyalkylene oxide blocks comprise on average 4 to 50 alkylene oxide units, preferably ethylene oxide units, per molecule.

Suitable sorbitan fatty acid esters are sorbitan monolaurate, sorbitan monopalmitate, sorbitan monostearate, sorbitan monooleate, sorbitan tristearate, sorbitan trioleate, sorbitan monostearate, sorbitan monolaurate or sorbitan monooleate.

Suitable polyalkoxylated sorbitan fatty acid esters are, for example, polyoxyethylene (20) sorbitan monolaurate, polyoxyethylene (20) sorbitan mono-palmitate, polyoxyethylene (20) sorbitan monostearate, polyoxyethylene (20) sorbitan monooleate, polyoxyethylene (20) sorbitan tristearate, polyoxyethylene (20) sorbitan trioleate, polyoxyethylene (4) sorbitan monostearate, polyoxyethylene (4) sorbitan monolaurate or polyoxyethylene (4) sorbitan monooleate.

Suitable polyalkoxylated glycerides are obtained e.g. by alkoxylation of natural or hydrogenated glycerides or by transesterification of natural or hydrogenated glycerides with polyalkylene glycols. Commercially available examples are polyoxyethylene glycerol ricinoleate-35, polyoxyethylene glycerol trihydroxy-stearate-40 (Cremophor® RH40, BASF AG) and polyalkoxylated glycerides, such as are obtainable from Gattefosse under the trade names Gelucire® and Labrafil®, e.g. Gelucire® 44/14 (lauroyl macrogol-32 glycerides, prepared by transesterification of hydrogenated palm kernel oil with PEG 1500), Gelucire® 50/13 (stearoyl macrogol-32 glycerides, prepared by transesterification of hydrogenated palm oil with PEG 1500) or Labrafil M1944 CS (oleoyl macrogol-6 glycerides, prepared by transesterification of apricot kernel oil with PEG 300).

A suitable fatty acid ester of polyalkylene glycols is e.g. PEG-660-hydroxystearic acid (polyglycol ester of 12-hydroxystearic acid (70 mol %) with 30 mol % ethylene glycol).

Suitable polyalkoxylated ethers of fatty alcohols are e.g. macrogol-6 cetyl stearyl ether or macrogol-25 cetyl stearyl ether.

Solubilizers are typically co-used in the powder mixture in an amount of from 0.1 to 15 wt. %, preferably 0.5 to 10 wt. %. Solubilizers having at least one polyalkoxy unit in the molecule are preferred.

Disintegrating agents, such as crosslinked polyvinylpyrrolidone and crosslinked sodium carboxymethylcellulose,

Extenders or fillers, such as lactose, cellulose, silicates or silica,

Lubricants, such as magnesium stearate and calcium stearate, sodium stearyl fumarate,

Dyestuffs, such as azo dyestuffs, organic or inorganic pigments or dyestuffs of natural origin,

Stabilizers, such as antioxidants, light stabilizers, agents which destroy hydroperoxide, agents which trap free radicals, stabilizers against microbial attack.

For the preparation of the solid dosage forms, a melt, i.e. a shapeable cohesive mass, is prepared from the powder mixture at an elevated temperature, i.e. a temperature at or above the softening point of the binder, and is then cooled, optionally after a shaping section. Preferably, the time span over which the components are exposed to the elevated temperature is less than 5 minutes for each of the components, in particular less than 3 minutes.

The mixing of the components to give the powder mixture is carried out in conventional mixers, such as ploughshare mixers, vibratory or gravity mixers and the like. For the preparation of the powder mixture, it is also possible to prepare a premix of individual components and/or part amounts of the components and to mix in the remaining components and/or the remaining amounts of individual components at a later point in time. Thus, for example, a premix which is free from active compound can be kept in stock and one or more active compounds can be mixed in as required. Alternatively, it may be advantageous to prepare a premix which comprises the active compounds and auxiliaries and optionally a part amount of the binder and to incorporate this premix into the binder and/or the main amount of the binder.

The heating is carried out in a conventional device for this purpose. Heatable extruders of kneaders, such as mixing/kneading reactors (e.g. ORP, CRP, AP, DTB from List or Reactotherm from Krauss-Maffei or co-kneaders from Buss), double-bowl kneaders (trough mixers) and plunger kneaders (internal mixers) or rotor/stator systems (e.g. Dispax from IKA) are particularly suitable.

Extruders which can be employed are single-screw machines, combing screw machines or also multi-shaft extruders, in particular twin-screw extruders rotating in the same direction or opposite directions and optionally equipped with kneading disks. Twin-screw extruders of the ZSK model series from Werner u. Pfleiderer are particularly preferred.

The extruder or kneader is charged continuously or discontinuously in the conventional manner, depending on the design thereof. The powder mixture is preferably introduced in a free feed, e.g. via a differential metering balance.

The melt obtained is dough-like to paste-like. It is as a rule subjected to shaping. A large number of shapes can be produced here, depending on the mould and the nature of the shaping. For example, if an extruder is used, the extruded strand can be shaped between a belt and a roll, between two belts or between two rolls, as described in EP-A-358 105, or by calendering in a calender with two shaping rolls, see, for example, EP-A-240 904. Small-particled granules can be obtained, for example, by extrusion and hot or cold chopping off of the strand.

The cooled masses can subsequently also be ground to powders and then pressed to tablets in the conventional manner. In this context, tabletting auxiliaries, such as colloidal silica, calcium hydrogen phosphate, lactose, microcrystalline cellulose, starch or magnesium stearate, can be co-used.

The invention is illustrated in more detail by the following examples.

### EXAMPLES 1 TO 3

10.5 parts by wt. of copovidone were mixed with 2 parts by wt. of solubilizer (Labrafil M 1944 CS) in a laboratory mixing granulator (Bohle) in the course of 5 min. The granules obtained were mixed with 71.5 parts by wt. of copovidone, 15.0 parts by wt. of fenofibrate and 1.0 part by wt. of highly disperse silica (Aerosil 200) in the course of 40 min. Aliquots of the powder mixture were mixed homogeneously with the amounts of titanium dioxide stated in Table 1 (anatase; min. 98%<10 μm) in a vibratory mixer (TURBULA) in the course of 2.5 min; the properties of the mixtures obtained are summarized in Table 1, and the particle size distribution determined by sieve analysis is summarized in Table 2 (the percentage content which remains using a sieve having the stated mesh width is stated). The bulk density was determined by means of a JEL tamping volumeter from Engelsmann, Ludwigshafen, Germany.

TABLE 1

| | Titanium dioxide | Bulk density | Angle of repose |
|---|---|---|---|
| Example | [wt. %] | [g/ml] | [°] |
| 1 (Comparison) | 0 | 0.27 | 40.14 |
| 2 | 1 | 0.29 | 38.30 |
| 3 | 5 | 0.34 | 35.34 |

TABLE 2

| | | | | | | | 1,000 |
|---|---|---|---|---|---|---|---|
| Example | Pan | 100 μm | 200 μm | 315 μm | 500 μm | 630 μm | μm |
| 1 | 0.3 | 9.3 | 85.6 | 3.7 | 0.5 | 0.3 | 0.5 |
| 2 | 36.4 | 52.1 | 9.0 | 2.0 | 0.1 | 0.2 | 0 |
| 3 | 53.9 | 35.4 | 8.7 | 2.3 | 0.4 | 0.3 | 0 |

The feed hopper of a twin-screw extruder (ZSK 25; Werner & Pfleiderer) was filled with the powder mixtures and extrusion was carried out at an extrusion temperature of 80-155° C. The throughput was 5 kg/h in Example 1 and 7-8 kg/h in Example 3. The extrudate was cut into pieces and allowed to solidify. The extrudate pieces were ground in a high-speed mill (Comil). The ground material (97.64 parts by wt.) was mixed with sodium stearyl fumarate (1.30 parts by wt.) and highly disperse silica (Aerosil 200; 1.06 parts by wt.) in a TURBULA mixer in the course of 5 min. Tablets of 54 mg or 160 mg active compound content were prepared from the mixture on an eccentric tablet press (Fette E 1).

The dissolving rates of the resulting tablets in a 0.05 M solution of sodium dodecyl sulfate (75 rpm) are summarized in Tables 3A and 3B.

TABLE 3A
Dissolving of active compound [%] 54 mg tablet

| Example | 10 min | 20 min | 30 min | 40 min | 50 min | 60 min |
|---|---|---|---|---|---|---|
| 1 | 26.5 | 45.4 | 60.8 | 76.6 | 87.1 | 92.6 |
| 2 | 25.8 | 45.5 | 61.4 | 78.7 | 87.6 | 98.3 |
| 3 | 26.1 | 49.1 | 67.8 | 84.5 | 96.8 | 100 |

TABLE 3B
Dissolving of active compound [%] 160 mg tablet

| Example | 10 min | 20 min | 30 min | 40 min | 50 min | 60 min |
|---|---|---|---|---|---|---|
| 1 | 10.5 | 25.8 | 36.3 | 50.4 | 62.1 | 75.6 |
| 2 | 18.6 | 34.0 | 46.7 | 59.3 | 69.1 | 85.3 |
| 3 | 26.9 | 44.9 | 56.6 | 69.7 | 79.2 | 88.0 |

It can be seen that the dissolving rate increases with an increasing content of titanium dioxide, the effects being more pronounced at a higher tablet weight.

### EXAMPLES 4 To 6

Examples 1 to 3 were repeated, hydroxypropylmethylcellulose (HPMC) being used instead of the copovidone. The properties of the powder mixtures obtained are summarized in Table 4.

TABLE 4

| | Titanium dioxide | Bulk density | Angle of repose |
|---|---|---|---|
| Example | [wt. %] | [g/ml] | [°] |
| 4 (Comparison) | 0 | 0.35 | 46.49 |
| 5 | 1 | 0.36 | 43.43 |
| 6 | 5 | 0.38 | 41.99 |

### EXAMPLE 7

150.0 g of fenofibrate, 715.0 g of the matrix polymer mentioned in Table 5, 125.0 g of Labrafil M 1944 CS and 10 g of Aerosil 200 were mixed homogeneously in a vibratory mixer (TURBULA) in the course of 2 min at 30 rpm. The premix obtained in this way was sieved over a 2 mm sieve (Frewitt S1B). The end mixture was mixed in a vibratory mixer (TURBULA) for 3 min at 30 rpm. Corresponding mixtures which contained 10.0 g or 50.0 g of titanium dioxide were furthermore prepared; the amount of matrix polymer was reduced by this amount. The properties of the powder mixtures obtained are summarized in Table 5.

TABLE 5
Angle of repose of powder mixtures

| Tita- | | | | | | | |
|---|---|---|---|---|---|---|---|
| nium | | | | | | | |
| diox- | | | | | | K30/ | K30/ |
| ide | | Maize | | Macrogol | | HPMC | HPMC |
| wt. % | HPC | starch | Poloxamer | 6000 | K30 | 1:1 | 2:1 |
| 0 | 44.0 | 47.8 | 32.3 | 44.7 | 32.5 | 36.7 | 35.4 |
| 1 | 42.2 | 44.7 | 30.0 | 44.2 | 32.2 | 37.0 | 34.5 |
| 5 | 40.4 | 43.0 | 33.2 | 42.5 | 29.1 | 35.0 | 32.9 |

HPC: hydroxypropylcellulose
K30: polyvinylpyrrolidone (Fikentscher K value 30)
HPMC: hydroxypropylmethylcellulose
119/119

1. A solid dosage form comprising a matrix with at least one active compound dispersed homogeneously in the matrix, which is obtainable by melting a powder mixture, wherein the powder mixture comprises at least one thermoplastic binder and a combination of highly disperse silica and at least one inorganic pigment.

2. The dosage form as claimed in claim 1, wherein the active compound is present in the matrix in the form of a solid solution.

3. The dosage form as claimed in claim 1, wherein the powder mixture comprises a tinctorially active amount of the inorganic pigment.

4. The dosage form as claimed in claim 1, wherein the inorganic pigment is chosen from titanium dioxide and iron oxides.

5. The dosage form as claimed in claim 4, wherein the titanium dioxide is present in the anatase modification.

6. The dosage form as claimed in claim 1, wherein the inorganic pigment has an average particle size of less than 50 μm.

7. The dosage form as claimed in claim 1, wherein the powder mixture comprises 0.8 to 5.0 wt. % of inorganic pigment.

8. The dosage form as claimed in claim 1, wherein the powder mixture comprises 0.5 to 2.0 wt. % of highly disperse silica.

9. The dosage form as claimed in claim 1, wherein the powder mixture furthermore comprises at least one solubilizer.

10. The dosage form as claimed in claim 9, wherein the solubilizer contains at least one polyalkoxy unit.

11. The dosage form as claimed in preceding claim 1, wherein the thermoplastic binder is chosen from sugar alcohols, derivatives thereof and water-soluble and water-dispersible polymers.

12. The dosage form as claimed in claim 11, wherein the thermoplastic binder is chosen from homo- or copolymers of vinylpyrrolidone, polyalkylene oxides, starch, hydroxypropylcellulose, hydroxypropylmethylcellulose and mixtures thereof.

13. A process for the preparation of solid dosage forms, in which a powder mixture which comprises at least one thermoplastic binder and a combination of highly disperse silica and at least one inorganic pigment is provided, the powder mixture is melted, and the melt obtained is shaped into dosage forms.

14. The process as claimed in claim 13, in which the cooled melt is ground to particles and the particles are pressed to dosage forms.
